(19) United States
(12) Patent Application Publication
(10) Pub. No.: US 2022/0313600 A1
(43) Pub. Date: Oct. 6, 2022

(54) FORMULATIONS OF PSMA IMAGING AGENTS

(71) Applicant: Clarity Pharmaceuticals Limited, Eveleigh (AU)

(72) Inventors: Paul Stephen DONNELLY, Parkville (AU); Nicholas Alan ZIA, Victoria (AU); Lawson Kyle SPARE, Eveleigh (AU); Ellen Marianne VAN DAM, Eveleigh (AU); Kevin Kar Weng KUAN, Eveleigh, NSW (AU)

(73) Assignee: Clarity Pharmaceuticals Limited, Eveleigh (AU)

(21) Appl. No.: 17/594,991

(22) PCT Filed: May 22, 2020
(86) PCT No.: PCT/AU2020/050509

§ 371 Date: Nov. 4, 2021

(30) Foreign Application Priority Data
May 24, 2019 (AU) 2019901765

(51) Int. Cl. A61K 9/00; A61K 51/04; A61K 47/12; A61K 47/20; C07F 1/08

(52) U.S. Cl. CPC A61K 9/0029; A61K 51/0482; A61K 47/12; A61K 51/0427; A61K 47/20; C07F 1/08

(57) ABSTRACT

The present invention relates to formulations of radiolabeled compounds that are of use in radiotherapy and diagnostic imaging related to prostate specific membrane antigen (PSMA).

### FIELD

The present invention relates to formulations of radiolabeled compounds that are of use in radiotherapy and diagnostic imaging related to prostate specific membrane antigen (PSMA).

### BACKGROUND

Prostate cancer is a leading cause of cancer-related deaths in men, with the mortality rate often attributed to difficulties in the detection and subsequent treatment of the disease. Prostate-related tumours often show increased expression of prostate-specific membrane antigen (PSMA), which is an enzyme typically expressed in prostate tissue but is often upregulated in some prostate cancers. This means that PSMA is a good biomarker or target for imaging, diagnostic, prognostic purposes. However since PSMA is also expressed in other tissues, both normal and malignant, difficulties exist in successfully imaging prostate cancer.

Radiolabelled complexes may be used for the imaging and treatment of cancers such as prostate cancer, however some complexes containing a radioisotope or radionuclide and a targeting ligand may be unstable and prone to dissociation. Where the complex formed is not sufficiently strong, dissociation may occur shortly after formation, i.e. during the radiolabeling process. While processes for radiolabeling are known, these may not result in the complex being formed in sufficient yield, or the overall solution of the complex may not be radiochemically pure. Furthermore, even if the radiolabelled complex could be produced, purification and isolation procedures that allow for the intact complex to be obtained in good yield are preferred.

Even if the radiolabelled complex may be obtained, the complex may be unstable and prone to degradation. This may result in the dissociation of the radioisotope, reduced radiochemical yield and purity of the formulation containing the complex and limited efficiency of the formulation. Where the radioisotope is lost and not delivered to the intended cancer site, imaging and/or treatment is either of a reduced quality or insufficient.

The radiolabelled complex may also be prone to radiolysis, where the activity of the radioisotope leads to destruction and degradation of the ligand owing to the spontaneous decay of the radioisotope. This leads to release of the radioisotope. Diffusion of the free radioisotope to other areas as a result of the circulatory system may result in the delivery of radioactivity to locations where it is not desired.

There exists a need for stable formulations of radiolabelled complexes that are suitable for the imaging and treatment of prostate cancer. There is also a requirement for effective processes for preparing such stable formulations.

### SUMMARY

In one aspect of the present invention, there is provided an aqueous formulation for parenteral administration comprising a compound of Formula (I), or a salt thereof, complexed with a Cu ion:

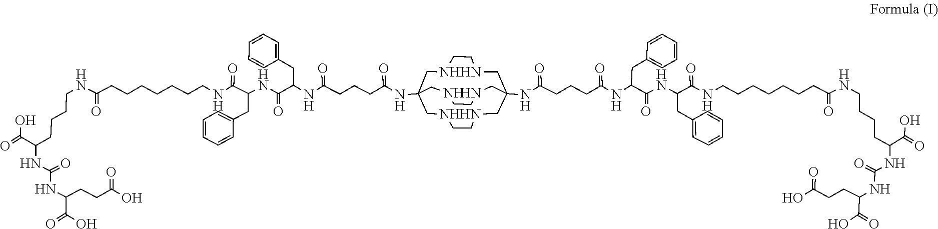 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the formulation further comprising at least one of gentisic acid, ascorbic acid, L-methionine, pyridoxine or a salt thereof.

In a further aspect, there is provided an aqueous formulation for parenteral administration comprising a compound of Formula (Ia), or a salt thereof, complexed with a Cu ion:

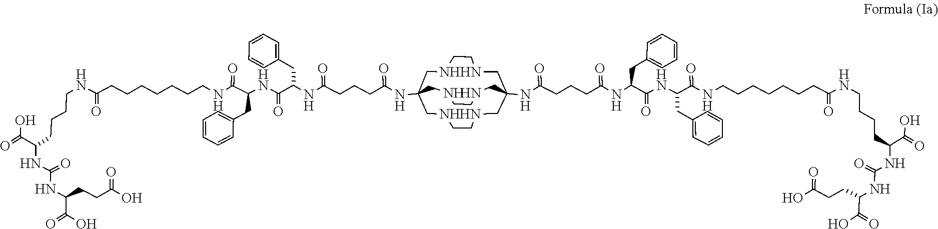

the formulation further comprising at least one of gentisic acid, ascorbic acid, L-methionine, pyridoxine or a salt thereof.

In another aspect of the present invention, there is provided an aqueous formulation for parenteral administration comprising a compound of Formula (I), or a salt thereof, complexed with a Cu ion:

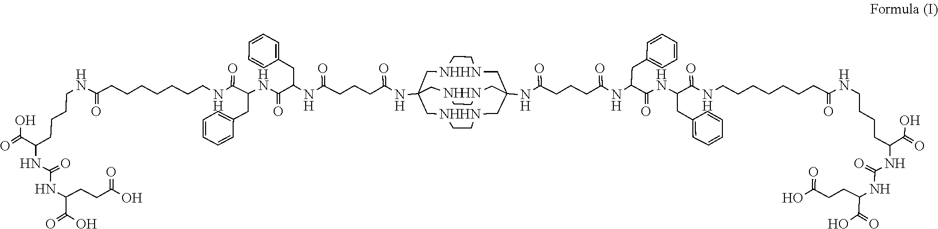 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the formulation further comprising a buffer solution.

In a further aspect, there is provided an aqueous formulation for parenteral administration comprising a compound of Formula (Ia), or a salt thereof, complexed with a Cu ion:

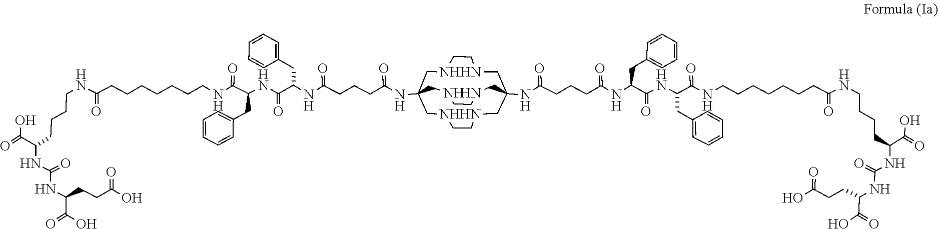

the formulation further comprising a buffer solution.

In an embodiment, the aqueous formulation comprises gentisic acid, or a salt thereof.

In another embodiment, the aqueous formulation comprises ascorbic acid, or a salt thereof.

In another embodiment, the aqueous formulation comprises L-methionine or a salt thereof.

In another aspect of the present invention, there is provided a process for preparing a formulation comprising a compound of Formula (I) complexed with a Cu radioisotope,

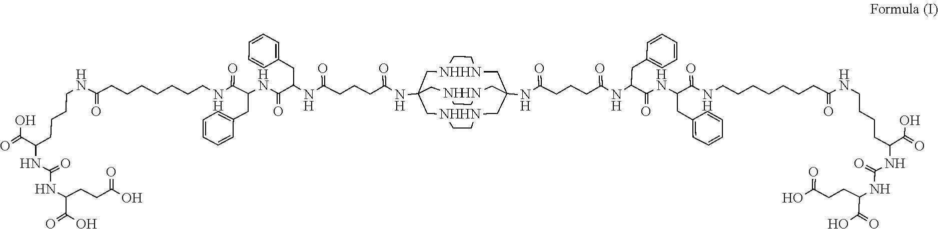 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) adding an amount of a compound of Formula (I) to an acetate buffer;
ii) adding a solution of a Cu radioisotope in hydrochloric acid to the compound of Formula (I) and the acetate buffer; and
iii) heating the mixture of step ii) for a time and under conditions for a complex of Formula (I) and the Cu radioisotope to form.

In another aspect of the present invention, there is provided a process for preparing a formulation comprising compound of Formula (I) complexed with a Cu radioisotope,

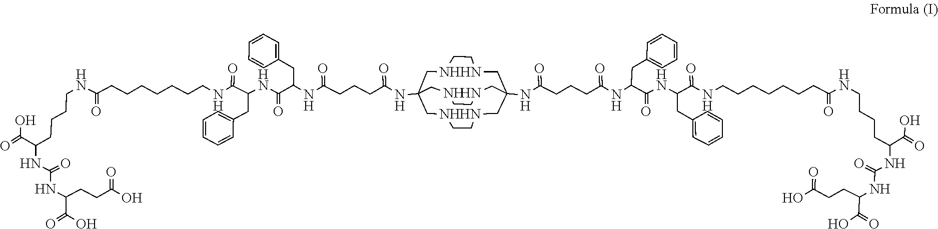 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) adding an amount of a compound of Formula (I) to a phosphate buffer;
ii) adding a solution of a Cu radioisotope in hydrochloric acid to the compound of Formula (I) and the phosphate buffer; and
iii) allowing the mixture of step ii) to react for a time and under conditions for a complex of Formula (I) and the Cu radioisotope to form.

In an embodiment of the process for preparing the complex as defined herein, the compound of Formula (I) has the structure of the compound of Formula (Ia):

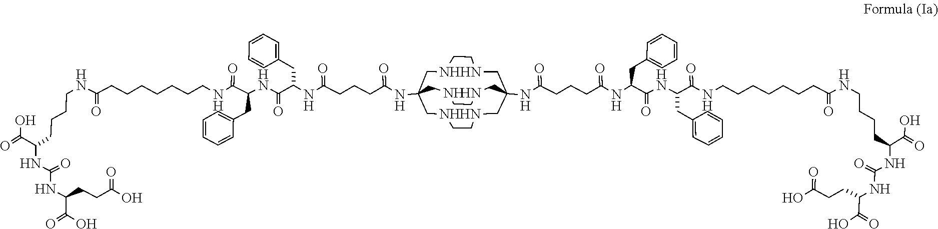

In an embodiment, the Cu radioisotope is ⁶¹Cu.

In an embodiment, the Cu radioisotope is ⁶⁴Cu.

In another embodiment, the Cu radioisotope is ⁶⁷Cu.

In a further embodiment, the pH of the formulation is maintained in the range between about 4 and about 8.

In another aspect of the present invention, there is provided a process for purifying a compound of Formula (I) complexed with a Cu radioisotope:

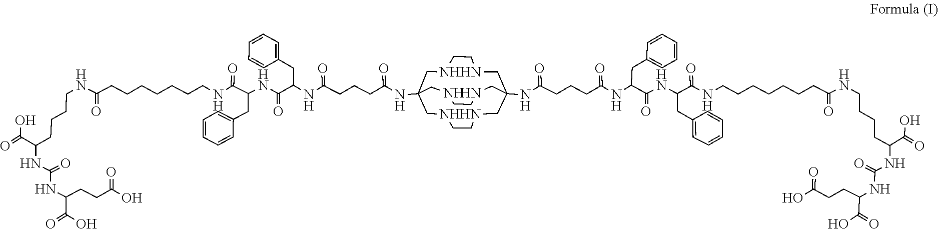 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) loading a solution of a compound of Formula (I) complexed with a Cu radioisotope on to a solid phase extraction cartridge;
ii) eluting the compound of Formula (I) complexed with a Cu radioisotope with an eluent comprising water, ethanol and sodium chloride.

In an embodiment of the process for purifying the complex as defined herein, the compound of Formula (I) has the structure of the compound of Formula (Ia):

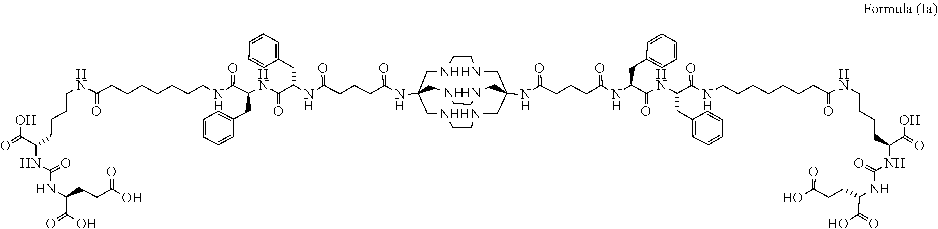

In an embodiment, the purified compound of Formula (I), or a salt thereof, complexed with a Cu radioisotope purified according to an earlier aspect is prepared according to another aspect of the present invention.

In an embodiment, the compound of Formula (I), or a salt thereof, complexed with a Cu radioisotope is prepared according to another aspect of the present invention.

### BRIEF DESCRIPTION OF THE FIGURES

FIG. 1: Radiochemical purity of a solution of the purified complex of Formula (Ia) and ⁶⁴Cu, where the solution contains either gentisic acid, ascorbic acid or L-methionine as monitored over a period of 48 hours.

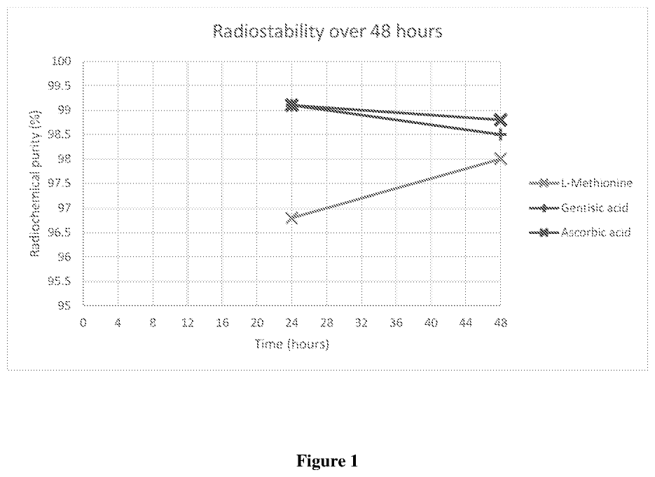

### DETAILED DESCRIPTION

### Formulations of Complexes of Formula (I)

The present invention relates to stable formulations of a specific radioisotope-ligand complex. The present inventors have found that the formulations of a complex disclosed herein minimise dissociation of the radioisotope from the ligand and/or minimise radiolysis of the ligand arising from the radioisotope.

The formulations of a radioisotope-ligand complex referred to herein are stable in solution and under physiological conditions for a time. The stability of the formulation relates to the stability of the complex. The radioisotope may undergo dissociation from the complex, which leads to less radioactivity being delivered to the site to which the ligand binds. Since the radioisotope undergoes spontaneous decay or the emission of energy, this energy when emitted may lead to degradation of the ligand, which is termed radiolysis. The radiostability of the complex can be measured by considering the radiochemical purity of the formulation. Radiochemical purity is defined as the amount of the radioisotope complexed by the sarcophagine ligand expressed as percentage of the total amount of the radioisotope present in the formulation. The radioisotope may be present in the formulation as a complex with the sarcophagine ligand, as a free radioisotope or as part of a radiolysis product.

It has previously been found that ligands containing a urea-based motif are known to bind to the catalytic site of prostate-specific membrane antigen (PSMA), which is typically expressed in prostate tissue and upregulated in some prostate cancers. An example of a ligand containing such a motif is Sar-bisPSMA, which is the macrocyclic ligand 1,8-diamino-3,6,10,13,16,19-hexaazabicyclo[6.6.6.]icosane (also known as sarcophagine or “Sar”), where each terminal amine group is attached to a linker group and a urea-based motif.

Sar-bisPSMA is shown in Formula (I):

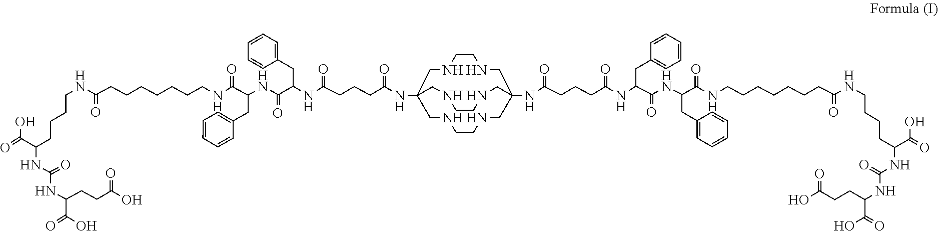 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

The compound of Formula (I) may be produced through a series of coupling reactions between the sarcophagine ligand, the linker and the urea motif. Procedures for the preparation of the compound of Formula (I) can be found in WO 2018/223180.

The compound of Formula (I) may have the structure of Formula (Ia) as depicted below, where the stereochemistry of compound is defined:

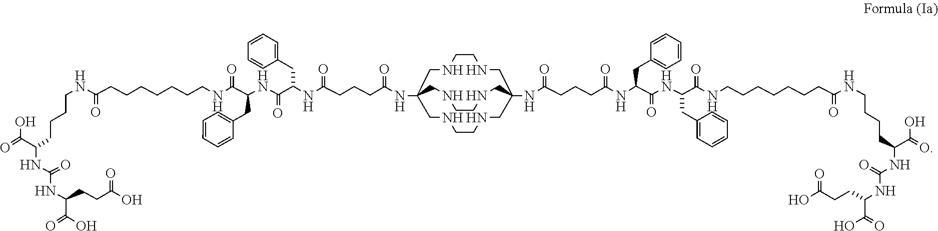

Where unspecified, any reference to the compound of Formula (I) below should be taken to include a reference to the compound of Formula (Ia) as well.

The present invention is related to the use of the compounds of Formula (I) and (Ia) in formulations. The compounds of Formula (I) and (Ia) may be used as a pharmaceutically acceptable salt. The compounds of Formula (I) and (Ia) contain two urea motifs that are independently capable of binding to the catalytic site of PSMA. The present inventors believe that the increased binding affinity of compounds of Formula (I) at the desired site is due to the presence of a second urea motif. Without wishing to be bound by theory, the present inventors believe that the additional binding affinity of compounds of Formula (I), which appears to be more effective than the use of twice the amount of the analogous compound having only one urea motif, is related to the presence of the second urea motif. Subsequently, the formulations described herein containing a compound of Formula (I) or (Ia) show better efficacy than formulations of an analogous compound containing the urea motif.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) and (Ia) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

In a preferred embodiment, the compound of Formula (I) is provided as an acetate salt.

The formulations of the present invention comprise a compound of Formula (I), or a salt thereof, and a radioisotope. The radioisotope, which may also be referred to as a radionuclide, may be a metal or a metal ion. The compound of Formula (I) of the present specification has been found to be particularly successful in complexing copper ions, especially Cu²⁺ ions. A person skilled in the art would appreciate that a complex of the compound of Formula (I) may be produced by contacting the compound of Formula (I) with the desired radioisotope, where the radioisotope is a Cu²⁺ ion.

In an embodiment, the ligand is complexed with a Cu ion. The copper ion may be radioactive, and thus a radionuclide or radioisotope of copper. In an embodiment, the ligand is complexed with ⁶⁰Cu. In another embodiment, the ligand is complexed with ⁶¹Cu. In another embodiment, the ligand is complexed with ⁶⁴Cu. In another embodiment, the ligand is complexed with ⁶⁷Cu. In a preferred embodiment, the ligand is complexed with ⁶⁴Cu. In another preferred embodiment, the ligand is complexed with ⁶⁷Cu.

The complex of Formula (I) with a Cu radioisotope may be unstable and prone to radiolysis when in solution. The present inventors have found that the solubilised complex may be stabilised when one or more stabilising agents are added to the formulation comprising the complex. Such stabilising agents include gentisic acid, ascorbic acid, L-methionine, pyridoxine and salts thereof.

The formulations of the present invention may comprise at least one of gentisic acid, ascorbic acid, L-methionine and pyridoxine, or salts thereof. The present inventors have identified that the addition of gentisic acid, ascorbic acid, L-methionine and/or pyridoxine to the formulations of the present invention assist in preventing or minimising radiolysis of the complex of Formula (I), thus increasing the radiostability of the complex and formulation thereof.

Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant. Other isomers of dihydroxybenzoic acid are also contemplated. Examples of other isomers include 2,4-dihydroxybenzoic acid and 2,5-dihydroxybenzoic acid, and salts thereof.

In an embodiment, gentisic acid is present in the formulation in an amount of about 0.02% to about 0.1% (w/v). In an embodiment, gentisic acid, or a salt thereof, is present in the formulation in an amount of about 0.02% (w/v). In another embodiment, gentisic acid, or a salt thereof, is present in the formulation in an amount of about 0.025% (w/v). In another embodiment, gentisic acid, or a salt thereof, is present in the formulation in an amount of about 0.03% (w/v). In another embodiment, gentisic acid, or a salt thereof, is present in the formulation in an amount of about 0.035% (w/v). In another embodiment, gentisic acid, or a salt thereof, is present in the formulation in an amount of about 0.04% (w/v). In another embodiment, gentisic acid, or a salt thereof, is present in the formulation in an amount of about 0.045% (w/v). In another embodiment, gentisic acid, or a salt thereof, is present in the formulation in an amount of about 0.05% (w/v). In another embodiment, gentisic acid, or a salt thereof, is present in the formulation in an amount of about 0.055% (w/v). In another embodiment, gentisic acid, or a salt thereof, is present in the formulation in an amount of about 0.6% (w/v). In another embodiment, gentisic acid, or a salt thereof, is present in the formulation in an amount of about 0.065% (w/v). In another embodiment, gentisic acid, or a salt thereof, is present in the formulation in an amount of about 0.07% (w/v). In another embodiment, gentisic acid, or a salt thereof, is present in the formulation in an amount of about 0.075% (w/v). In another embodiment, gentisic acid, or a salt thereof, is present in the formulation in an amount of about 0.08% (w/v).). In another embodiment, gentisic acid, or a salt thereof, is present in the formulation in an amount of about 0.085% (w/v). In another embodiment, gentisic acid, or a salt thereof, is present in the formulation in an amount of about 0.09% (w/v). In another embodiment, gentisic acid, or a salt thereof, is present in the formulation in an amount of about 0.095% (w/v). In another embodiment, gentisic acid, or a salt thereof, is present in the formulation in an amount of about 0.1% (w/v). In other embodiments, the present invention also contemplates gentisic acid, or a salt thereof, in ranges between the aforementioned amounts. In a preferred embodiment, gentisic acid, or a salt thereof, is present in the formulation in an amount of not more than 0.056% (w/v).

L-Methionine is an amino acid comprising a thiol ether sidechain and is also known as Met or L-Met. Salts of L-methionine include the sodium salt. Any reference to L-methionine may include a reference to salts thereof, where relevant.

In an embodiment, L-methionine, or a salt thereof, is present in the formulation in an amount of about 1 mg/mL to about 4 mg/mL. In an embodiment, L-methionine, or a salt thereof, is present in the formulation in an amount of about 1.0 mg/mL. In another embodiment, L-methionine, or a salt thereof, is present in the formulation in an amount of about 1.5 mg/mL. In another embodiment, L-methionine, or a salt thereof, is present in the formulation in an amount of about 2.0 mg/mL. In another embodiment, L-methionine, or a salt thereof, is present in the formulation in an amount of about 2.5 mg/mL. In another embodiment, L-methionine, or a salt thereof, is present in the formulation in an amount of about 3.0 mg/mL. In another embodiment, L-methionine, or a salt thereof, is present in the formulation in an amount of about 3.5 mg/mL. In another embodiment, L-methionine, or a salt thereof, is present in the formulation in an amount of about 4.0 mg/mL. In other embodiments, the present invention also contemplates L-methionine, or a salt thereof, in ranges between the aforementioned amounts. In a preferred embodiment, L-methionine is present in the formulation in an amount of about 3 mg/mL.

Ascorbic acid is also known as 2,3-didehydro-L-threo-hexano-1,4-lactone or Vitamin C. Salts of ascorbic acid include sodium ascorbate, potassium ascorbate, calcium ascorbate and magnesium ascorbate. Any reference to ascorbic acid may include a reference to salts thereof, where relevant.

In an embodiment, ascorbic acid, or a salt thereof, is present in the formulation in an amount of about 5 mg/mL to about 50 mg/mL. In an embodiment, ascorbic acid, or a salt thereof, is present in the formulation in an amount of about 5 mg/mL. In another embodiment, ascorbic acid, or a salt thereof, is present in the formulation in an amount of about 6 mg/mL. In another embodiment, ascorbic acid, or a salt thereof, is present in the formulation in an amount of about 7 mg/mL. In another embodiment, ascorbic acid, or a salt thereof, is present in the formulation in an amount of about 8 mg/mL. In another embodiment, ascorbic acid, or a salt thereof, is present in the formulation in an amount of about 9 mg/mL. In another embodiment, ascorbic acid, or a salt thereof, is present in the formulation in an amount of about 10 mg/mL. In another embodiment, ascorbic acid, or a salt thereof, is present in the formulation in an amount of about 11 mg/mL. In another embodiment, ascorbic acid, or a salt thereof, is present in the formulation in an amount of about 12 mg/mL. In another embodiment, ascorbic acid, or a salt thereof, is present in the formulation in an amount of about 13 mg/mL. In another embodiment, ascorbic acid, or a salt thereof, is present in the formulation in an amount of about 14 mg/mL. In another embodiment, ascorbic acid, or a salt thereof, is present in the formulation in an amount of about 15 mg/mL. In another embodiment, ascorbic acid, or a salt thereof, is present in the formulation in an amount of about 20 mg/mL. In another embodiment, ascorbic acid, or a salt thereof, is present in the formulation in an amount of about 25 mg/mL. In another embodiment, ascorbic acid, or a salt thereof, is present in the formulation in an amount of about 30 mg/mL. In another embodiment, ascorbic acid, or a salt thereof, is present in the formulation in an amount of about 35 mg/mL. In another embodiment, ascorbic acid, or a salt thereof, is present in the formulation in an amount of about 40 mg/mL. In another embodiment, ascorbic acid, or a salt thereof, is present in the formulation in an amount of about 45 mg/mL. In another embodiment, ascorbic acid, or a salt thereof, is present in the formulation in an amount of about 50 mg/mL. In other embodiments, the present invention also contemplates ascorbic acid, or a salt thereof, in ranges between the aforementioned amounts. In a preferred embodiment, ascorbic acid is present in the formulation in an amount of about 10 mg/mL.

Pyridoxine is also known as 4,5-bis(hydroxymethyl)-2-methylpyridin-3-ol or Vitamin B6. Salts of pyridoxine may include the hydrochloride salt.

In an embodiment, pyridoxine, or a salt thereof, is present in the formulation in an amount of about 5 mg/mL to about 15 mg/mL. In an embodiment, pyridoxine, or a salt thereof, is present in the formulation in an amount of about 5 mg/mL. In another embodiment, pyridoxine, or a salt thereof, is present in the formulation in an amount of about 6 mg/mL. In another embodiment, pyridoxine, or a salt thereof, is present in the formulation in an amount of about 7 mg/mL. In another embodiment, pyridoxine, or a salt thereof, is present in the formulation in an amount of about 8 mg/mL. In another embodiment, pyridoxine, or a salt thereof, is present in the formulation in an amount of about 9 mg/mL. In another embodiment, pyridoxine, or a salt thereof, is present in the formulation in an amount of about 10 mg/mL. In another embodiment, pyridoxine, or a salt thereof, is present in the formulation in an amount of about 11 mg/mL. In another embodiment, pyridoxine, or a salt thereof, is present in the formulation in an amount of about 12 mg/mL. In another embodiment, pyridoxine, or a salt thereof, is present in the formulation in an amount of about 13 mg/mL. In another embodiment, pyridoxine, or a salt thereof, is present in the formulation in an amount of about 14 mg/mL. In another embodiment, pyridoxine, or a salt thereof, is present in the formulation in an amount of about 15 mg/mL. In a preferred embodiment, pyridoxine is present in the formulation in an amount of about 10 mg/mL.

The formulations of the present invention may comprise ethanol as a component. The ethanol used in the formulation may be anhydrous ethanol. Alternatively, the ethanol used in the formulation may not have been subject to drying processes and may be hydrated. The ethanol is preferably pharmaceutical grade ethanol. The ethanol present in the formulation may further assist in preventing radiolysis of the radiolabelled complex of Formula (I).

In an embodiment, ethanol is present in the formulation in an amount of about 7% to about 13% (v/v). In an embodiment, ethanol is present in the formulation in an amount of about 7% (v/v). In another embodiment, ethanol is present in the formulation in an amount of about 8% (v/v). In another embodiment, ethanol is present in the formulation in an amount of about 9% (v/v). In another embodiment, ethanol is present in the formulation in an amount of about 10% (v/v). In another embodiment, ethanol is present in the formulation in an amount of about 11% (v/v). In another embodiment, ethanol is present in the formulation in an amount of about 12% (v/v). In another embodiment, ethanol is present in the formulation in an amount of about 13% (v/v). In a preferred embodiment, ethanol is present in the formulation in an amount of about 10% (v/v). In other embodiments, the present invention also contemplates ethanol in ranges between the aforementioned amounts.

The formulations of the present invention may also comprise sodium chloride as a component.

The sodium chloride in the formulations of the present invention may be provided as a saline solution. A saline solution is defined as an aqueous solution of sodium chloride. For example, normal saline is defined as an aqueous solution of sodium chloride at a concentration of 0.9% (w/v). In an embodiment of the present invention, the sodium chloride of a formulation is provided by a saline solution.

In an embodiment, sodium chloride is present in the formulation in an amount of about 0.6% to 1.2% (w/v). In an embodiment, sodium chloride is present in an amount of about 0.6% (w/v). In another embodiment, sodium chloride is present in an amount of about 0.7% (w/v). In another embodiment, sodium chloride is present in an amount of about 0.8% (w/v). In another embodiment, sodium chloride is present in an amount of about 0.9% (w/v). In another embodiment, sodium chloride is present in an amount of about 1.0% (w/v). In another embodiment, sodium chloride is present in an amount of about 1.1% (w/v). In another embodiment, sodium chloride is present in an amount of about 1.2% (w/v). In a preferred embodiment, sodium chloride is present in the formulation in an amount of about 0.9% (w/v). In other embodiments, the present invention also contemplates sodium chloride in ranges between the aforementioned amounts.

The formulations of the present invention have a pH of about 4 to about 8. A person skilled in the art would understand that the pH of the formulation is an inherent characteristic of the formulation, attributed to the combination of the compound of Formula (I) or a complex thereof, and the remaining excipients of the formulation. Alternatively, the pH of the formulation may be modified to the desired value by the addition of one or more buffering agents. An examples of a suitable buffer solution includes an acetate buffer, which may comprise a mixture of sodium acetate and acetic acid. In certain embodiments, formulations of the present invention comprise an acetate buffer. Another suitable buffer solution includes a phosphate buffer, which may comprise a mixture of various phosphate salts or hydrates thereof. Examples of phosphate salts that are suitable include sodium dihydrogen phosphate (NaH₂PO₄), disodium hydrogen phosphate (Na₂HPO₄), potassium dihydrogen phosphate (KH₂PO₄) and dipotassium hydrogen phosphate (K₂HPO₄). In an embodiment, the phosphate buffer contains sodium phosphate salts. In another embodiment, the phosphate buffer contains potassium phosphate salts. In another embodiment, the phosphate buffer contains a mixture of sodium and potassium phosphate salts.

As used herein, the term “buffer” refers to a component that maintains the pH of the medium to which it is added at a constant level. In the context of the present disclosure, gentisic acid, ascorbic acid, L-methionine and pyridoxine, their salts or aqueous solutions thereof are not considered to be buffers.

In an embodiment, the pH of the formulation is from about 4 to about 8. In an embodiment, the pH of the formulation is about 4. In another embodiment, the pH of the formulation is about 4.5. In another embodiment, the pH of the formulation is about 5.0. In an embodiment, the pH of the formulation is about 5.5. In another embodiment, the pH of the formulation is about 5.6. In another embodiment, the pH of the formulation is about 5.7. In another embodiment, the pH of the formulation is about 5.8. In another embodiment, the pH of the formulation is about 5.9. In another embodiment, the pH of the formulation is about 6.0. In another embodiment, the pH of the formulation is about 6.1. In another embodiment, the pH of the formulation is about 6.2. In another embodiment, the pH of the formulation is about 6.3. In another embodiment, the pH of the formulation is about 6.4. In another embodiment, the pH of the formulation is about 6.5. In another embodiment, the pH of the formulation is about 7.0. In another embodiment, the pH of the formulation is about 7.5. In another embodiment, the pH of the formulation is about 8.0. In a preferred embodiment, the pH of the formulation is about 6.0. In another preferred embodiment, the pH of the formulation is about 5.0.

In the hands of the present inventors, when the compound of Formula (I) is formulated as an aqueous solution, it was identified that the compound was relatively unstable and prone to oxidation and degradation. One approach to overcome the observed instability may be to add one or more components that are antioxidants and/or stabilizing agents to the formulation, however the inclusion of further components to a formulation introduces potential reactivity issues between the compound of Formula (I) and these added components. For instance, the addition of an antioxidant may in fact react with the compound of Formula (I) thus potentially changing the structure and function of the compound, which is undesirable.

The present inventors have now found that although the addition of particular stabilizing agents may, in some cases, be sufficient to provide a formulation containing the compound of Formula (I). As disclosed herein, formulations of compounds of Formula (I) containing a stabilizing agent, such as gentisic acid, ascorbic acid, L-methionine or pyridoxine, are contemplated, since they do not appear to react with the compound of Formula (I) and can provide the requisite stability.

However it has now been found that a formulation containing a buffer and a compound of Formula (I) provides the requisite stability to the compound. Thus the present inventors have found that the presence of a buffer, in addition to providing a formulation with an appropriate pH for parenteral administration, provides a formulation where the compound of Formula (I) has the requisite stability.

Surprisingly, the inventors have found that although the addition of a stabilizing agent such as gentisic acid, ascorbic acid and other agents discussed herein may provide the required stability, stability of the formulation may also be achieved by the use of a buffer alone, i.e. in the absence of the stabilizing agent.

### Processes for Preparing Complexes of Formula (I)

The present invention also relates to processes for preparing a radiolabelled complex of compounds of Formula (I) and formulations thereof. As previously discussed compounds of Formula (I) may be complexed with a radioisotope, such as a Cu ion. Accordingly, the present invention provides a process for preparing a formulation comprising a compound of Formula (I) complexed with a Cu radioisotope:

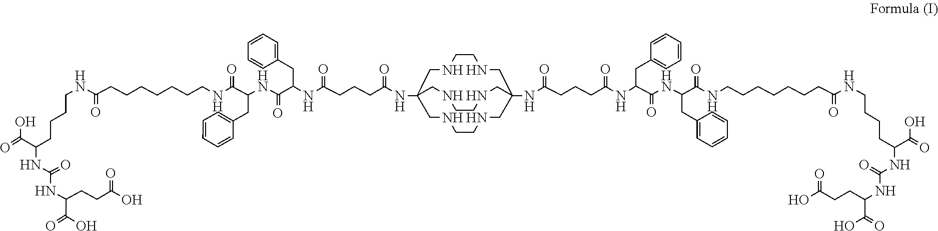 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) adding an amount of a compound of Formula (I) to an acetate buffer;
ii) adding a solution of a Cu radioisotope in hydrochloric acid to the compound of Formula (I) and the acetate buffer; and
iii) heating the mixture of step ii) for a time and under conditions for a complex of Formula (I) and the Cu radioisotope to form.

The present invention also provides a process for preparing a formulation comprising a compound of Formula (I) complexed with a Cu radioisotope:

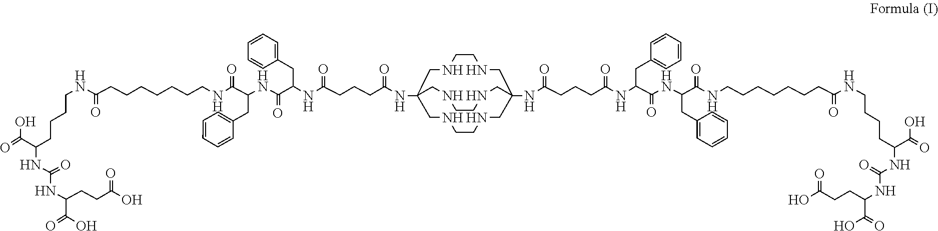 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) adding an amount of a compound of Formula (I) to a phosphate buffer solution;
ii) adding a solution of a Cu radioisotope in hydrochloric acid to the compound of Formula (I) and the phosphate buffer solution; and
iii) allowing the mixture of step ii) to react for a time and under conditions for a complex of Formula (I) and the Cu radioisotope to form.

In certain embodiments, the compound of Formula (I) has the structure of the compound of Formula (Ia):

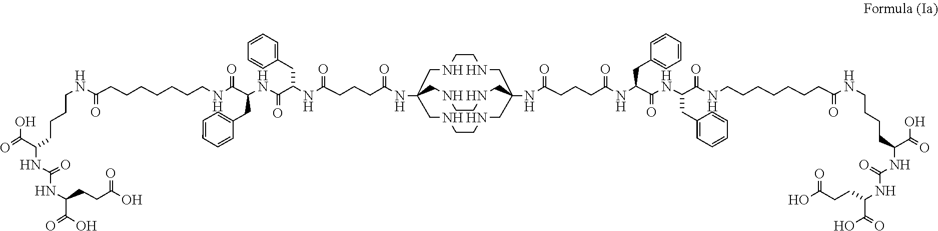

In an embodiment, the process further includes the step adding a solution of sodium ascorbate to the mixture of the compound of Formula (I) and the Cu radioisotope, once the reaction between the compound of Formula (I) and the Cu radioisotope is complete.

The compound of Formula (I) may be provided as part of a stock solution. Prior to preparation of the stock solution of Formula (I), the compound may be subject to drying steps, such as lyophilisation. The compound of Formula (I) may be dissolved in a mixture of ethanol and water to create a stock solution of the compound of Formula (I). In an embodiment, the compound of Formula (I) is dissolved in a mixture of ethanol and water, where the ethanol and water is present in a ratio of about 1:1. In an embodiment, the compound of Formula (I) is provided as a stock solution at a concentration of about 1 nmol/μL.

The compound of Formula (I) may be present in an amount between about 1 nmol and about 10 nmol. In an embodiment, the compound of Formula (I) is present in an amount of about 1 nmol. In another embodiment, the compound of Formula (I) is present in an amount of about 2 nmol. In another embodiment, the compound of Formula (I) is present in an amount of about 3 nmol. In another embodiment, the compound of Formula (I) is present in an amount of about 4 nmol. In another embodiment, the compound of Formula (I) is present in an amount of about 5 nmol. In another embodiment, the compound of Formula (I) is present in an amount of about 6 nmol. In another embodiment, the compound of Formula (I) is present in an amount of about 7 nmol. In another embodiment, the compound of Formula (I) is present in an amount of about 8 nmol. In another embodiment, the compound of Formula (I) is present in an amount of about 9 nmol. In another embodiment, the compound of Formula (I) is present in an amount of about 10 nmol. One skilled in the art would understand that the required volume of a stock solution of the compound Formula (I) depends on the initial concentration of the stock solution. One skilled in the art would also understand that larger amounts of the compound of Formula (I) may be used and subsequently the amounts of the other reagents, buffers and solvents may be modified accordingly.

In an embodiment, the buffer solution may be an acetate buffer. The acetate buffer used in the process may be prepared from sodium acetate and acetic acid. The acetate buffer maintains the pH in a range suitable for complexation of the compound of Formula (I) with the Cu radioisotope. The pH of the buffer solution may be about 5.0. The acetate buffer may have a concentration of about 1.0 M. The acetate buffer may also comprise ethanol. In an embodiment, the acetate buffer comprises ethanol in an amount between about 10% and about 30%. In an embodiment, the acetate buffer comprises ethanol in an amount of about 10%. In an embodiment, the acetate buffer comprises ethanol in an amount of about 20%. In an embodiment, the acetate buffer comprises ethanol in an amount of about 30%. In a preferred embodiment, the acetate buffer comprises ethanol in an amount of about 20%.

In another embodiment, the buffer solution may be a phosphate buffer. The phosphate buffer may comprise a mixture of various phosphate salts or hydrates thereof. Examples of phosphate salts that are suitable include sodium dihydrogen phosphate (NaH₂PO₄), disodium hydrogen phosphate (Na₂HPO₄), potassium dihydrogen phosphate (KH₂PO₄) and dipotassium hydrogen phosphate (K₂HPO₄). In an embodiment, the phosphate buffer contains sodium phosphate salts. In another embodiment, the phosphate buffer contains potassium phosphate salts. In another embodiment, the phosphate buffer contains a mixture of sodium and potassium phosphate salts. The phosphate buffer may also contain saline and/or water. In an embodiment, the phosphate buffer comprises a mixture of sodium hydrogen phosphate salts and saline.

From a stock solution of the compound of Formula (I) in a mixture of ethanol and water, an aliquot containing an amount of the compound of Formula (I) is taken and mixed with an amount of the acetate buffer. In an embodiment, the compound of Formula (I) is added to the acetate buffer, wherein the acetate buffer comprises about 20% ethanol. In an embodiment, the compound of Formula (I) is added to the acetate buffer at room temperature.

As discussed above, the compound of Formula (I) complexes Cu ions. In an embodiment, the Cu ion is a radioisotope of Cu. In an embodiment, the Cu radioisotope is ⁶⁰Cu. In another embodiment, the Cu radioisotope is ⁶¹Cu. In another embodiment, the Cu radioisotope is ⁶⁴Cu. In another embodiment, the Cu radioisotope is ⁶⁷Cu. The Cu radioisotope is provided as a Cu salt. In an embodiment, the Cu salt is provided as a Cu²⁺ chloride salt. In an embodiment, the Cu salt is provided as a [⁶⁴Cu]CuCl₂ salt. The Cu radioisotope is provided as a solution of hydrochloric acid. In an embodiment, the Cu radioisotope is ⁶⁴Cu and is provided as a solution of hydrochloric acid, where the hydrochloric acid has a concentration of about 0.02 M. In an embodiment, the Cu radioisotope is provided as a solution of [⁶⁴Cu]CuCl₂ in a solution of hydrochloric acid, where the hydrochloric acid has a concentration of about 0.02 M. One skilled in the art would understand that the Cu salt may be provided in other concentrations of hydrochloric acid. In another embodiment, the Cu radioisotope is provided as a Cu²⁺ acetate salt. In an embodiment, the Cu salt is provided as a [⁶⁴Cu]Cu(OAc)₂ salt.

The solution of the Cu salt provided as a hydrochloric acid solution will have a particular starting radioactivity. The starting activity of the solution may vary, depending on the particular batch of the radioisotope. One skilled in the art would understand that the final activity of the compound of Formula (I) complexed with a Cu ion will depend on the activity of the Cu salt used to complex the compound of Formula (I) and that this will in turn depend on the activity of the solution of the Cu salt in hydrochloric acid. The overall radiochemical yield of the complex of Formula (I) and the copper salt may be determined with respect to the amount of the radioactivity initially present in the solution of the Cu salt. An aliquot of the Cu radioisotope in a solution of hydrochloric acid is added to the compound of Formula (I) in the acetate buffer. One skilled in the art would understand that radiochemical purity may be determined by radioHPLC or a similar method.

In an embodiment, the solution of the ⁶⁴Cu radioisotope has a radioactivity of between about 100 and about 5000 MBq. In an embodiment, the solution of the ⁶⁴Cu radioisotope has a radioactivity of about 100 MBq. In another embodiment, the solution of the ⁶⁴Cu radioisotope has a radioactivity of about 250 MBq. In another embodiment, the solution of the ⁶⁴Cu radioisotope has a radioactivity of about 500 MBq. In another embodiment, the solution of the ⁶⁴Cu radioisotope has a radioactivity of about 750 MBq. In another embodiment, the solution of the ⁶⁴Cu radioisotope has a radioactivity of about 1000 MBq. In another embodiment, the solution of the ⁶⁴Cu radioisotope has a radioactivity of about 1500 MBq. In another embodiment, the solution of the ⁶⁴Cu radioisotope has a radioactivity of about 2000 MBq. In another embodiment, the solution of the ⁶⁴Cu radioisotope has a radioactivity of about 2500 MBq. In another embodiment, the solution of the ⁶⁴Cu radioisotope has a radioactivity of about 3000 MBq. In another embodiment, the solution of the ⁶⁴Cu radioisotope has a radioactivity of about 4000 MBq. In another embodiment, the solution of the ⁶⁴Cu radioisotope has a radioactivity of about 5000 MBq.

In an embodiment, the solution of the ⁶¹Cu radioisotope has a radioactivity of between about 100 and about 5000 MBq. In an embodiment, the solution of the ⁶¹Cu radioisotope has a radioactivity of about 100 MBq. In another embodiment, the solution of the ⁶¹Cu radioisotope has a radioactivity of about 250 MBq. In another embodiment, the solution of the ⁶¹Cu radioisotope has a radioactivity of about 500 MBq. In another embodiment, the solution of the ⁶¹Cu radioisotope has a radioactivity of about 750 MBq. In another embodiment, the solution of the ⁶¹Cu radioisotope has a radioactivity of about 1000 MBq. In another embodiment, the solution of the ⁶¹Cu radioisotope has a radioactivity of about 1500 MBq. In another embodiment, the solution of the ⁶¹Cu radioisotope has a radioactivity of about 2000 MBq. In another embodiment, the solution of the ⁶¹Cu radioisotope has a radioactivity of about 2500 MBq. In another embodiment, the solution of the ⁶¹Cu radioisotope has a radioactivity of about 3000 MBq. In another embodiment, the solution of the ⁶¹Cu radioisotope has a radioactivity of about 4000 MBq. In another embodiment, the solution of the ⁶¹Cu radioisotope has a radioactivity of about 5000 MBq.

In an embodiment, the solution of the ⁶⁷Cu radioisotope has a radioactivity of between about 100 and about 3000 MBq. In an embodiment, the solution of the ⁶⁷Cu radioisotope has a radioactivity of about 100 MBq. In another embodiment, the solution of the ⁶⁷Cu radioisotope has a radioactivity of about 250 MBq. In another embodiment, the solution of the ⁶⁷Cu radioisotope has a radioactivity of about 500 MBq. In another embodiment, the solution of the ⁶⁷Cu radioisotope has a radioactivity of about 750 MBq. In another embodiment, the solution of the ⁶⁷Cu radioisotope has a radioactivity of about 1000 MBq. In another embodiment, the solution of the ⁶⁷Cu radioisotope has a radioactivity of about 1500 MBq. In another embodiment, the solution of the ⁶⁷Cu radioisotope has a radioactivity of about 2000 MBq. In another embodiment, the solution of the ⁶⁷Cu radioisotope has a radioactivity of about 2500 MBq. In another embodiment, the solution of the ⁶⁷Cu radioisotope has a radioactivity of about 3000 MBq. In another embodiment, the solution of the ⁶⁷Cu radioisotope has a radioactivity of about 4000 MBq. In another embodiment, the solution of the ⁶⁷Cu radioisotope has a radioactivity of about 5000 MBq.

The Cu radioisotope may be provided as a solution in hydrochloric acid. In an embodiment, the Cu radioisotope is provided in a hydrochloric acid solution having a concentration of between about 0.01 M and about 0.05 M. In an embodiment, the concentration of the hydrochloric acid solution is about 0.01 M. In another embodiment, the concentration of the hydrochloric acid solution is about 0.02 M. In another embodiment, the concentration of the hydrochloric acid solution is about 0.03 M. In another embodiment, the concentration of the hydrochloric acid solution is about 0.04 M. In another embodiment, the concentration of the hydrochloric acid solution is about 0.05 M. In a further embodiment, the concentration of the hydrochloric acid solution is between about 0.02 M and about 0.05 M.

The solution comprising the mixture of the Cu radioisotope, the compound of Formula (I) and the acetate buffer is then allowed to mix for a time and at a particular temperature in order to allow for complexation of the compound of Formula (I) with the Cu radioisotope. The solution may be mixed using an appropriate apparatus. For example, given the small volumes used, an Eppendorf tube may be an appropriate vessel, such that an Eppendorf Thermomixer may be used to both mix and if required, heat the solution. In an embodiment, the solution is mixed at room temperature. In an embodiment, the solution is mixed at about 40° C. The present inventors have found that complexation of the radioisotope is complete within about 5 minutes when the solution is mixed at a temperature of about 40° C. While a lower temperature of about 21° C., i.e. room temperature, may be used for mixing, the complexation reaction may not be complete at 5 minutes but is completed by about 15 minutes. The present inventors have also found that temperatures higher than about 40° C., for example 60° C., leads to some degradation of the compound of Formula (I) thus reducing the yield of the complex. In an embodiment, the solution is mixed at about 40° C. for about 5 minutes. In an embodiment, the solution is mixed at about 40° C. for about 10 minutes. In another embodiment, the solution is mixed at about 40° C. for about 15 minutes. In another embodiment, the solution is mixed at about 21° C. for about 10 minutes. In another embodiment, the solution is mixed at about 21° C. for about 15 minutes.

In certain embodiments, the compound of Formula (I) is added to a phosphate buffer solution, to which a solution containing the Cu radioisotope in hydrochloric acid is added. The mixture containing the compound of Formula (I), the phosphate buffer and the Cu radioisotope is allowed to react for a time and under conditions so as to provide a complex of Formula (I) and the Cu radioisotope. In an embodiment, the solution is mixed at room temperature. In an embodiment, the solution is mixed at room temperature for about 10 minutes. In another embodiment, the solution is mixed at room temperature for about 15 minutes. In another embodiment, the solution is mixed at room temperature for about 20 minutes. In a further embodiment, the solution is mixed at room temperature for about 25 minutes.

Once the complex of Formula (I) and the Cu radioisotope is formed, the solution is diluted with a sodium ascorbate solution. The addition of sodium ascorbate introduces a reducing agent to the mixture, which in turn provides a radiostabilising effect to the complex of Formula (I) and the Cu radioisotope. In turn, this increases the stability of the formulation as a whole and allows for a longer shelf life of the formulation containing the complex. The sodium ascorbate solution may have a concentration of between about 25 mg/mL and about 75 mg/mL. In an embodiment, the sodium ascorbate solution may have a concentration of about 25 mg/mL. In another embodiment, the sodium ascorbate solution may have a concentration of about 50 mg/mL. In another embodiment, the sodium ascorbate solution may have a concentration of about 75 mg/mL. The sodium ascorbate solution of a particular concentration is added in an amount so as to ensure that any remaining Cu radioisotope is sufficiently diluted. A person skilled in the art would understand that the volume of the solution added will depend on the amount of uncomplexed Cu radioisotope and the concentration of the sodium ascorbate solution.

The present inventors have found that the process for preparing a compound of Formula (I) complexed with a Cu radioisotope as disclosed herein allows for efficient radiolabelling of the compound and allows for a high radiochemical yield to be obtained. The present inventors have found that the complexation of the compound of Formula (I) with the Cu radioisotope is faster if an acetate buffer comprising an amount of ethanol is used. According to an embodiment of the present invention, the process comprises adding the compound of Formula (I) to an acetate buffer comprising ethanol.

In an embodiment, the process comprises the steps of:
i) adding an amount of a compound of Formula (I) to an acetate buffer comprising ethanol;
ii) adding a solution of [⁶⁴Cu]CuCl₂ in hydrochloric acid to the compound of Formula (I) and the acetate buffer comprising ethanol; and
iii) heating the mixture of step ii) for about 5 minutes at 40° C.

In another embodiment, the process comprises the steps of:
i) adding an amount of a compound of Formula (I) to a phosphate buffer comprising ethanol and sodium gentisate;
ii) adding a solution of [⁶⁴Cu]CuCl₂ in hydrochloric acid to the compound of Formula (I) and the phosphate buffer comprising sodium gentisate; and
iii) allowing the mixture of step ii) to react for a time and under conditions for a complex of Formula (I) and the Cu radioisotope to form.

### Process for Purifying the Complex of Formula (I)

Once the process to prepare the compound of Formula (I) complexed with a Cu radioisotope is complete, the complex must be purified and isolated. One skilled in the art would understand that during purification and isolation processes, loss of material may occur thus reducing the overall chemical and radiochemical yield. These losses may be due to mechanical handling steps, loss of material in syringes and other apparatus used in the purification process or retention of material in the reaction vessel. The purification process typically involves a filtration step using a solid phase medium, retention of material on the solid phase often leads to reduced yield. The purification process often relies on washing of the solid phase with various solvents in order to elute the complex, however the use of large amounts of solvent leads to the dilute solutions of the complex which are undesirable. Degradation of the complex may also occur during purification, which may result in reduced yield of the complex and also loss of the free radioisotope.

The present inventors have now found that purification of the complex of Formula (I) and a Cu radioisotope is advantageously achieved, such that the complex is isolated in a high chemical and radiochemical yield.

According to another aspect of the present invention, there is provided a process for purifying a compound of Formula (I) complexed with a Cu radioisotope:

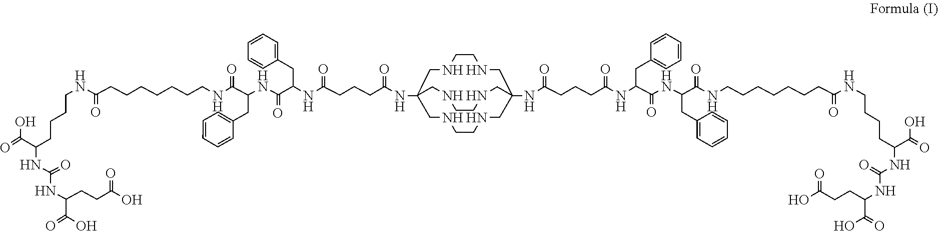 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]

the process comprising the steps of:
i) loading a solution of a compound of Formula (I) complexed with a Cu radioisotope on to a solid phase extraction cartridge;
ii) eluting the compound of Formula (I) complexed with a Cu radioisotope with an eluent comprising water, ethanol and sodium chloride.

In an embodiment of the process, the compound of Formula (I) has the structure of the compound of Formula (Ia):

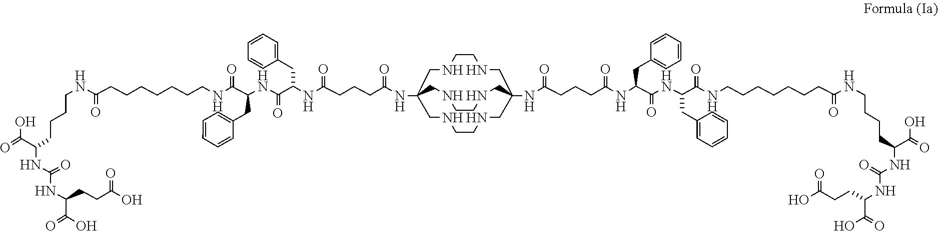

In an embodiment, the solution of a compound of Formula (I) complexed with a Cu radioisotope is obtained according to another embodiment of the present invention. Once the reaction to prepare a complex of Formula (I) and a Cu radioisotope is complete, the resultant solution is subjected to a purification process.

The solution comprising a complex of Formula (I) with a Cu radioisotope is loaded on to a solid phase extraction cartridge. The solid phase extraction cartridge contains a stationary phase that retains the complex and other components present in the solution. As used herein, the term “stationary phase” refers to a resin-like material that is held within the solid phase extraction cartridge and allows for the separation of compounds based on their polarity.

The solid phase extraction process as described herein may use a reverse-phase stationary phase. As used herein, the term “reverse-phase” in relation to a stationary phase refers to a stationary phase that is hydrophobic in nature, such that the stationary phase has an affinity for hydrophobic or uncharged molecules. Examples of a reverse-phase stationary phrase may include Waters Sep-Pak cartridges, such as C8, C18, light C18, light CN, light tC2 or HLB cartridges. Prior to loading of the solution containing the complex of Formula (I), the cartridge is primed by washing with ethanol, drying with air and equilibrating with water. In an embodiment, the solid phase extraction cartridge is a Waters C18 cartridge. In another embodiment, the solid phase extraction cartridge is a Waters tC2 cartridge. In another embodiment, the solid phase extraction cartridge is a Waters CN cartridge. In another embodiment, the solid phase extraction cartridge is a Waters HLB cartridge.

The purified solution containing the complex of Formula (I) with a Cu radioisotope may be subsequently used to produce a formulation comprising the complex. For example, one or more pharmaceutically acceptable diluents, adjuvants and/or excipients may be added to a solution containing the complex of Formula (I) with a Cu radioisotope. The diluents, adjuvants and excipients must be “acceptable” in terms of being compatible with the other ingredients of the composition, and not deleterious to the recipient thereof. Pharmaceutical carriers for preparation of pharmaceutical compositions are well known in the art, as set out in textbooks such as Remington's Pharmaceutical Sciences, 20^{th} Edition, Williams & Wilkins, Pennsylvania, USA. The carrier will depend on the route of administration, and again the person skilled in the art will readily be able to determine the most suitable formulation for each particular case.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### General Experimental Details

Sodium acetate buffers for radiolabelling were prepared using sodium acetate (TraceSELECT, Fluka, Batch #BCBM4793V), acetic acid (TraceSELECT, Fluka, Batch #BCBM5177V) and MilliQ water in acid-washed glass bottles and stored in acid-washed plastic bottles. All buffers were stored at 2-4° C. while not in use.

Phosphate buffers for radiolabelling were prepared using sodium phosphate dibasic (anhydrous), sodium phosphate monobasic and TraceSELECT water. All buffers were stored at room temperature while not in use.

Copper-64 (⁶⁴Cu) was obtained from SAHMRI, SA, Australia as [⁶⁴Cu]CuCl₂ in 0.02 M HCl, Batch #19-0075-902R, with a starting activity of 2.39 GBq @ 08:39 in 450 μL volume.

SPE purifications of the radiolabelled products were performed using Waters Sep-Pak Cartridges light C8 (Lot #: 002836047A). Cartridges were conditioned by washing with EtOH (10 mL), followed by a bolus of Air (3×10 mL), then equilibrated with MilliQ H2O (10 mL) and air (3×10 mL).

MeCN for HPLC (Honeywell, Lot #S1RA1H) and trifluoroacetic acid for HPLC (TFA, ReagentPlus, 99%, Sigma Aldrich, Lot #SHBG2783V), (+)-Sodium L-ascorbate (Sigma Aldrich, >99%, Lot #: BCBV4424), L-methionine (Sigma Aldrich, >99.5%, Lot #: BCBS2107V) and gentisic acid sodium salt hydrate (Sigma Aldrich, >99%, Lot #: MKCC2280) were used as received. All HPLC mobile phases were prepared prior to use, filtered (using a 0.45 μm aqueous or organic filter) and degassed via a combination of ultrasonic irradiation under vacuum for 10 minutes. All EtOH used was 100% Ethyl alcohol (Molecular biology grade). All syringes used were ‘B Braun Injekt-F’.

Elution buffer was prepared as a 1:1 EtOH:H₂O+0.9% NaCl.

All reaction vials were acid-washed prior to use. Plastic microcentrifuge tubes were filled with 4 M HCl and allowed to stand at least overnight, the 4 M HCl was removed and the vials thoroughly washed with MilliQ H₂O and oven dried at 50° C., after drying the vial were sealed to prevent further contamination. Glassware was acid-washed by soaking in 4 M HNO₃ for a minimum of 12 hours, after the 4 M HNO₃ was decanted into a suitable waste container the glassware was thoroughly washed with MilliQ H₂O and oven dried at 50° C., after drying the glassware were sealed to prevent further contamination.

A stock solution of Sar-bis(PSMA), i.e. the compound of Formula (Ia), in EtOH:H₂O (1:1) was prepared to give a solution with the compound of Formula (Ia) at a concentration of 1 nmol/μL.

### Radiolabelling of Formula (I) with a Cu Radioisotope

### Example 1

To an acid-washed 500 μL microcentrifuge tube was added the labelling buffer (acetate, 50 μL, 1 M, pH 5.0) followed by 10 μL of the Formula I stock solution. To the buffer solution was added [⁶⁴Cu]CuCl₂ in 0.02M HCl (25 μL, 116 MBq). The microcentrifuge tube was sealed and the radioactivity present in the reaction measured using a dose calibrator. The tube was transferred to an Eppendorf Thermomixer C and heated at 40° C. for 20 minutes. At 20 minutes the reaction was removed from the thermomixer and a sample (5 μL) was withdrawn from the reaction, diluted with 1:1 EtOH:H₂O (5 μL) and injected onto radioHPLC systems (QC1, 5 μL). The reaction mixture was at room temperature while the final analysis was undertaken to determine if the radiochemical yield was >95% taking 7 minutes.

### Example 2

To an acid-washed 500 μL microcentrifuge tube was added the labelling buffer (Acetate, 50 μL, 1 M, pH 5.0) followed by 5 μL of the Formula I stock solution. To the buffer solution was added [⁶⁴Cu]CuCl₂ in 0.02 M HCl (25 μL, 109 MBq). The microcentrifuge tube was sealed and the radioactivity present in the reaction measured using a dose calibrator. The tube was transferred to an Eppendorf Thermomixer C and heated at 40° C. for 20 minutes. At 20 minutes the reaction was removed from the thermomixer and a sample (5 μL) was withdrawn from the reaction, diluted with 1:1 EtOH:H₂O (5 μL) and injected onto radioHPLC systems (QC1, 5 μL). The reaction mixture was at room temperature while the final analysis was undertaken to determine if the radiochemical yield was >95% taking 7 minutes.

### Example 3

To an acid-washed 1500 μL microcentrifuge tube was added the labelling buffer (acetate, 600 μL, 1 M, pH 5.0) followed by 20 μL of the Formula I stock solution. To the buffer solution was added [⁶⁴Cu]CuCl₂ in 0.02 M HCl (300 μL, 1136 MBq). The microcentrifuge tube was sealed and the radioactivity present in the reaction measured using a dose calibrator. The tube was transferred to an Eppendorf Thermomixer C and heated at 40° C. for 20 minutes. At 20 minutes the reaction was removed from the thermomixer and a sample (5 μL) was withdrawn from the reaction, diluted with 1:1 EtOH:H₂O (5 μL) and injected onto radioHPLC systems (QC1, 5 μL). The reaction mixture was at room temperature while the final analysis was undertaken to determine if the radiochemical yield was >95% taking 7 minutes.

### Example 4

To an acid-washed 500 μL microcentrifuge tube was added the labelling buffer (20% EtOH in acetate, 100 μL, 1 M, pH 5.0) followed by 10 μL of the Formula I stock solution. To the buffer solution was added [⁶⁴Cu]CuCl₂ in 0.02 M HCl (50 μL, 183 MBq). The microcentrifuge tube was sealed and the radioactivity present in the reaction measured using a dose calibrator. The tube was transferred to an Eppendorf Thermomixer C and heated at 40° C. for 20 minutes. At 20 minutes the reaction was removed from the thermomixer and a sample (5 μL) was withdrawn from the reaction, diluted with 1:1 EtOH:H₂O (5 μL) and injected onto radioHPLC systems (QC1, 5 μL). The reaction mixture was at room temperature while the final analysis was undertaken to determine if the radiochemical yield was >95% taking 7 minutes.

### Example 5

To an acid-washed 500 μL microcentrifuge tube was added the labelling buffer (acetate, 100 μL, 1 M, pH 5.0) followed by 5 μL of the Formula I stock solution. To the buffer solution was added [⁶⁴Cu]CuCl₂ in 0.02 M HCl (50 μL, 174 MBq). The microcentrifuge tube was sealed and the radioactivity present in the reaction measured using a dose calibrator. The tube was transferred to an Eppendorf Thermomixer C and heated at 21° C. for 20 minutes. At 5 and 15 minutes, aliquots (5 μL) were taken to for analysis to determine if the reaction was complete. These samples were diluted with 1:1 EtOH:H₂O (5 μL) and injected onto radioHPLC systems (QC1, 5 μL). The reaction mixture was placed back in the thermomixer while the final analysis was undertaken to determine if the radiochemical yield was >95% taking 7 minutes.

### Example 6

To a solution of Sar-bis(PSMA) (50 μg, 24.8 nmol) in 0.1 M Na/Na phosphate buffer (5 mL) containing sodium gentisate (5 mg, 0.03 mmol) was added [⁶⁴Cu]CuCl₂ in 0.02 M-0.05 M HCl (NMT 500 μL, NMT 5000 MBq) at room temperature. The resulting mixture was allowed to react at room temperature for up to 25 min. Upon completion, the reaction mixture was quenched with 50 mg/mL sodium ascorbate solution (15 mL). The resulting mixture is then transferred through a vented 0.22 μm filter into a sterile vial to afford the compound of Formula (I) complexed with a ⁶⁴Cu radioisotope.

### Example 7

To a solution of Sar-bis(PSMA)(50 μg, 24.8 nmol) in 0.1 M Na/Na phosphate buffer (4.5 mL) containing sodium gentisate (5 mg, 0.03 mmol) and ethanol (neat, 0.5 mL) was added [⁶⁴Cu]CuCl₂ in 0.02 M-0.05 M HCl (NMT 500 μL, NMT 5000 MBq) at room temperature. The resulting mixture was allowed to react at room temperature for up to 25 min. Upon completion, the reaction mixture was quenched with 50 mg/mL sodium ascorbate solution (15 mL). The resulting mixture is then transferred through a vented 0.22 μm filter into a sterile vial to afford the compound of Formula (I) complexed with a ⁶⁴Cu radioisotope.

### Purification Procedures

### Example 8

The solution obtained in Example 1 was purified using a C8 SPE cartridge, the product was eluted in 0.5 mL of 1:1 EtOH:H₂O+0.9% NaCl. 62% of the product eluted from the SPE and was shown have high radiochemical purity of 94.3% with zero ‘free copper’. 4% was lost in the dilution/load syringe, 12% was stuck in the reaction vial and 9% was lost on the SPE.<1% were lost in the SPE load and wash steps.

### Example 9

The solution obtained in Example 2 was purified using a C8 SPE cartridge, the product was eluted in 0.5 mL of 1:1 EtOH:H₂O+0.9% NaCl. 73% of the product eluted from the SPE and was shown have high radiochemical purity of 94.3% with 0.2% ‘free copper’. 7% was lost in the dilution/load syringe, 1% was stuck in the reaction vial and 14% was lost on the SPE.<1% were lost in the SPE load and wash steps.

### Example 10

The solution obtained in Example 3 was purified using a C8 SPE cartridge, the product was eluted in 0.5 mL of 1:1 EtOH:H₂O+0.9% NaCl. 64% of the product eluted from the SPE and was shown have high radiochemical purity of 96.6% with 0.1% ‘free copper’. 4% was lost in the dilution/load syringe, 1.5% was stuck in the reaction vial.

### Example 11

The solution obtained in Example 4 was purified using a C8 SPE cartridge, the product was eluted in 0.5 mL of 1:1 EtOH:H₂O+0.9% NaCl. 71% of the product eluted from the SPE and was shown have high radiochemical purity of 96.3% with zero ‘free copper’. 6% was lost in the dilution/load syringe, 3% was stuck in the reaction vial and 15% was lost on the SPE.<1% were lost in the SPE load and wash steps.

### Example 12

The solution obtained in Example 5 was purified using a C8 SPE cartridge, the product was eluted in 0.5 mL of 1:1 EtOH:H₂O+0.9% NaCl. 59% of the product eluted from the SPE and was shown have high radiochemical purity of 96.9% with 0.1% ‘free copper’. 11% was lost in the dilution/load syringe, 7% was stuck in the reaction vial and 20% was lost on the SPE.<1% were lost in the SPE load and wash steps.

### Preparation of Formulations

### Example 13

Aliquots of the purified solution of Example 10 were taken and diluted with a mixture of ethanol in saline to give solutions with a final concentration of about 10% ethanol in saline. One of gentisic acid (0.63 mg/mL), ascorbic acid (10 mg/mL) or L-methionine (3 mg/mL) was added and the radiochemical purity of each sample was monitored over a period of 48 hours.

1. An aqueous formulation for parenteral administration comprising a compound of Formula (I), or a salt thereof, complexed with a Cu ion: 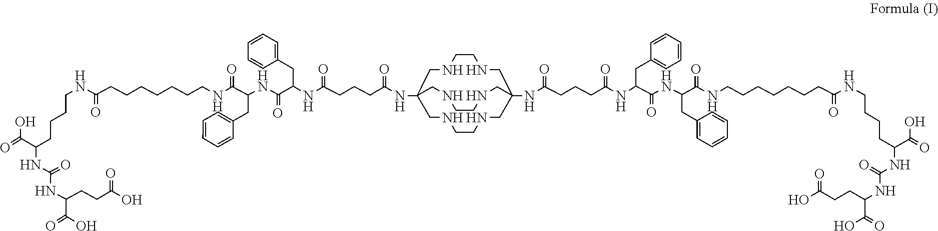 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]
the formulation further comprising at least one of gentisic acid, ascorbic acid, L-methionine, pyridoxine or a salt thereof; or an aqueous formulation for parenteral administration comprising a compound of Formula (I), or a salt thereof, complexed with a Cu ion: 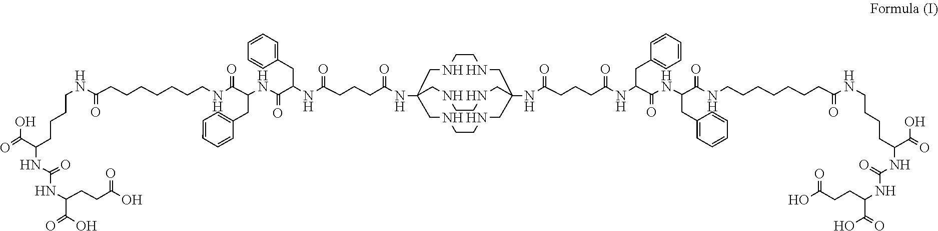 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]
the formulation further comprising a buffer solution.

2. (canceled)

3. An aqueous formulation according to claim 1, wherein the compound of Formula (I) has the structure of Formula (Ia):

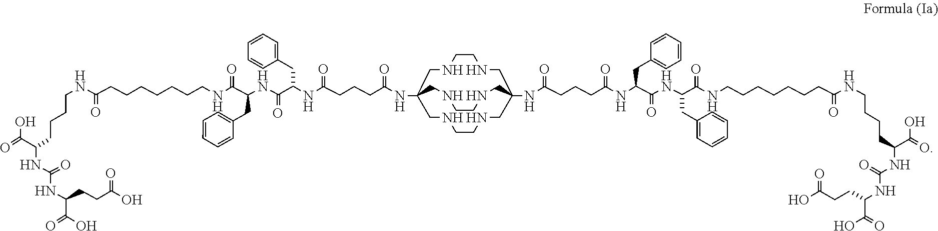

4. An aqueous formulation according to claim 1, wherein the formulation comprises gentisic acid, or a salt thereof.

5. An aqueous formulation according to claim 1, wherein the formulation comprises ascorbic acid, or a salt thereof.

6. An aqueous formulation according to claim 1, wherein the formulation comprises L-methionine, or a salt thereof.

7. An aqueous formulation according to claim 1, wherein the Cu ion is a Cu radioisotope.

8. An aqueous formulation according to claim 7, wherein the Cu radioisotope is ⁶⁴Cu.

9. An aqueous formulation according to claim 7, wherein the Cu radioisotope is ⁶⁷Cu.

10. An aqueous formulation according to claim 1, wherein the pH of the formulation is between about 4 to about 8.

11. A process for preparing a formulation comprising a compound of Formula (I) complexed with a Cu radioisotope, 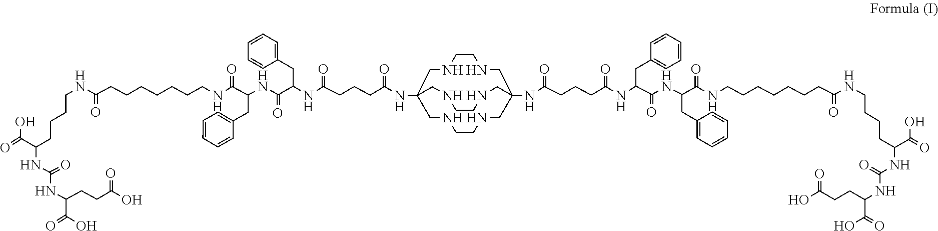 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]
the process comprising the steps of:
i) adding an amount of a compound of Formula (I) to an acetate buffer solution;
ii) adding a solution of a Cu radioisotope in hydrochloric acid to the compound of Formula (I) and the acetate buffer; and
iii) heating the mixture of step ii) for a time and under conditions for a complex of Formula (I) and the Cu radioisotope to form; or, process for preparing a formulation comprising a compound of Formula (I) complexed with a Cu radioisotope: 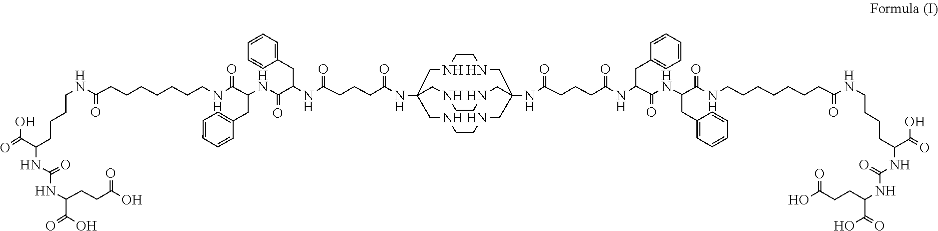 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]
the process comprising the steps of:
i) adding an amount of a compound of Formula (I) to a phosphate buffer solution;
ii) adding a solution of a Cu radioisotope in hydrochloric acid to the compound of Formula (I) and the phosphate buffer solution; and
iii) allowing the mixture of step ii) to react for a time and under conditions for a complex of Formula (I) and the Cu radioisotope to form.

12. (canceled)

13. A process according to claim 11, further comprising the step of:
iv) adding a solution of sodium ascorbate to the mixture of the compound of Formula (I) and the Cu radioisotope.

14. A process according to claim 11, wherein the compound of Formula (I) has the structure of Formula (Ia):

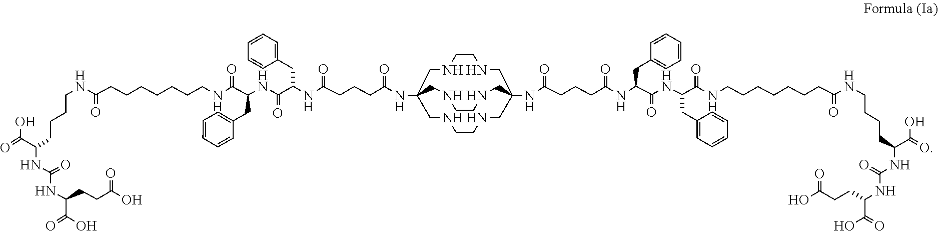

15. A process according to claim 11, wherein the Cu radioisotope is ⁶⁴Cu.

16. A process according to claim 11, wherein the Cu radioisotope is ⁶¹Cu.

17. A process according to claim 11, wherein the Cu radioisotope is ⁶⁷Cu.

18. A process according to claim 11, wherein the Cu radioisotope is provided as [⁶⁴Cu]CuCl₂.

19. A process according to claim 11, wherein the pH of the formulation is maintained in the range between about 4 and about 8.

20. A process for purifying a compound of Formula (I) complexed with a Cu radioisotope: 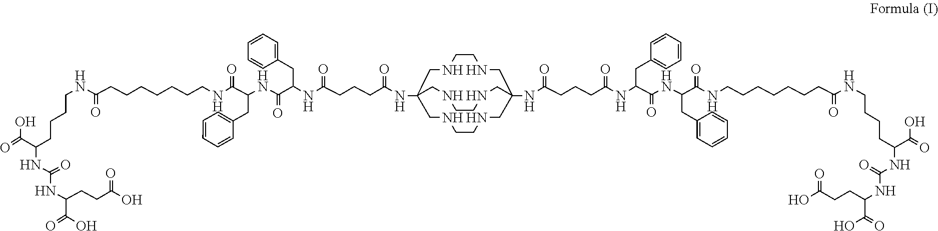 [SMILES: O=C(O)CCC(NC(=O)NC(CCCCNC(=O)CCCCCCCNC(=O)C(Cc1ccccc1)NC(=O)C(Cc1ccccc1)NC(=O)CCCC(=O)NC12CNCCNCC(NC(=O)CCCC(=O)NC(Cc3ccccc3)C(=O)NC(Cc3ccccc3)C(=O)NCCCCCCCC(=O)NCCCCC(NC(=O)NC(CCC(=O)O)C(=O)O)C(=O)O)(CNCCNC1)CNCCNC2)C(=O)O)C(=O)O]
the process comprising the steps of:
i) loading a solution of a compound of Formula (I) complexed with a Cu radioisotope on to a solid phase extraction cartridge;
ii) eluting the compound of Formula (I) complexed with a Cu radioisotope with an eluent comprising water, ethanol and sodium chloride.

21. A process according to claim 20, wherein the compound of Formula (I) has the structure of Formula (Ia): [chemical structure]
